(19) United States
(12) Patent Application Publication
(10) Pub. No.: US 2004/0265789 A1
(43) Pub. Date: Dec. 30, 2004

(54) Methods for the identification of inhibitors of 3-oxo-5-alpha-steroid 4-dehydrogenase expression or activity in plants

(76) Inventors: Robert Ascenzi, Cary, NC (US); Douglas Boyes, Chapel Hill, NC (US); Neil Hoffman, Bethesda, MD (US); Keith Davis, Durham, NC (US); Adel Zayed, Durham, NC (US); Rao Mulpuri, Apex, NC (US); Jeffrey Woessner, Hillsborough, NC (US); Jorn Gorlach, Manchester, NJ (US); Carol Hamilton, Apex, NC (US); Kenneth Phillips, Durham, NC (US)

Correspondence Address: Icoria, Inc., 108 T.W. ALEXANDER DRIVE, P O BOX 14528, RTP, NC, 27709-4528, US

(21) Appl. No.: 10/717,844

(22) Filed: Nov. 20, 2003

(30) Foreign Application Priority Data
May 30, 2002 (WO) PCT/US02/16786

(51) Int. Cl. C12Q 1/00

(52) U.S. Cl. 435/4

(57) ABSTRACT

The present inventors have discovered that 3-oxo-5-alpha-steroid 4-dehydrogenase (DET2) is essential for plant growth. Specifically, the inhibition of DET2 gene expression in plant seedlings resulted in seedlings that are shorter than controls and chlorotic. Thus, DET2 can be used as a target for the identification of herbicides. Accordingly, the present invention provides methods for the identification of compounds that inhibit DET2 expression or activity, comprising: contacting a compound with a DET2 and detecting the presence and/or absence of binding between said compound and said DET2, or detecting a decrease in DET2 expression or activity. The methods of the invention are useful for the identification of herbicides.

This application is the national phase under 35 U.S.C. § 371 of PCT International Application No. PCT/US02/16786, that has an International filing date of May 30, 2002, which designated the United States of America and which claims the benefit of U.S. Provisional Application No. 60/294,395, filed May 30, 2001.

### FIELD OF THE INVENTION

The invention relates generally to plant molecular biology. In particular, the invention relates to methods for the identification of herbicides.

### BACKGROUND OF THE INVENTION

3-oxo-5-alpha-steroid 4-dehydrogenase (EC 1.3.99.5) is a 5-alpha steroid which is implicated in the biosynthetic pathway of brassinosteroids, and which catalyzes the conversion of (24R)-24-methylcholest-4-en-3-one to (24R)-24-methyl-5alpha-cholestan-3-one. Other names for 3-oxo-5-alpha-steroid 4-dehydrogenase include steroid 5-alpha-reductase, DEETIOLATED2, and DET2. The Arabidopsis 3-oxo-5-alpha-steroid 4-dehydrogenase has been cloned and shown to encode a protein that shares approximately 40% sequence identity with mammalian steroid 5-alpha-reductases.

Noguchi et al. undertook further biochemical characterization of DET2 by using the det2 mutant, a plant line which contains a mutation of glutamate 204, thus abolishing the activity of DET2. Noguchi et al. (1999) Plant Physiol 120:833-40. The results of these studies indicated that the early operating steps of brassinosteroid biosynthesis are (24R)-24-methylcholest-4-en-3-one→(24R)-24-methyl-5alpha-cholestan-3-one in Arabidopsis, with the det2 mutant deficient in the conversion of 4-en-3-one to 3-one. This reaction is analogous to the conversion of testosterone to dihydrotestosterone in animals by 5-alpha-reductase. Both 5-alpha-reductase and DET2 are NADPH-dependent enzymes.

To date there do not appear to be any publications describing lethal effects of over-expression, antisense expression or knock-out of this gene in plants. Thus, the prior art has not suggested that DET2 is essential for plant growth and development. It would be desirable to determine the utility of this enzyme for evaluating plant growth regulators, especially herbicide compounds.

### SUMMARY OF THE INVENTION

The present inventors have discovered that antisense expression of a DET2 cDNA in Arabidopsis causes developmental abnormalities. Seedlings exhibited significant abnormalities, including being smaller and more chlorotic than controls. Thus, the present inventors have discovered that DET2 is essential for normal seed development and growth, and can be used as a target for the identification of herbicides. Accordingly, the present invention provides methods for the identification of compounds that inhibit DET2 expression or activity, comprising: contacting a candidate compound with a DET2 and detecting the presence or absence of binding between said compound and said DET2, or detecting a decrease in DET2 expression or activity. The methods of the invention are useful for the identification of herbicides.

### BRIEF DESCRIPTION OF THE DRAWINGS

FIG. 1 shows the 3-oxo-5-alpha-steroid 4-dehydrogenase (DET2) reaction.

### DETAILED DESCRIPTION OF THE INVENTION

### Definitions

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

The term “NADP⁺” refers to nicotinamide adenine dinucleotide phosphate, oxidized form.

The term “NADPH” refers to nicotinamide adenine dinucleotide phosphate, reduced form.

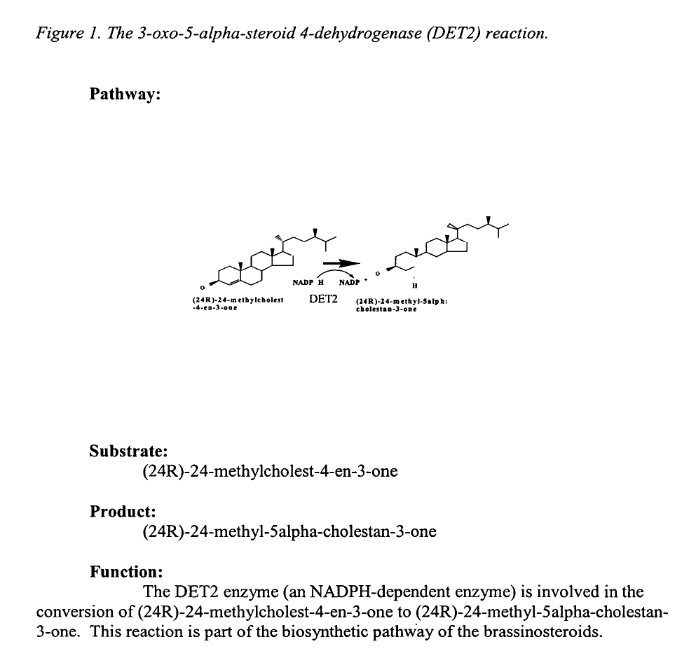

As used herein, the term “3-oxo-5-alpha-steroid 4-dehydrogenase” (EC 1.3.99.5) is synonymous with “DET2,” “DEETIOLATED2,” and “steroid 5-alpha-reductase,” and refers to an enzyme that catalyses the conversion of (24R)-24-methylcholest-4-en-3-one to (24R)-24-methyl-5alpha-cholestan-3-one during the biosynthesis of brassinosteroids, as shown in FIG. 1.

As used herein, the term “DNA” means deoxyribonucleic acid.

As used herein, the term “RNA” means ribonucleic acid.

As used herein, the term “mRNA” means messenger ribonucleic acid.

As used herein, the term “cDNA” means complementary deoxyribonucleic acid.

As used herein, the term “HPLC” means high pressure liquid chromatography.

As used herein, the term “TLC” means thin layer chromatography.

As used herein, the term “ELISA” means enzyme-linked immunosorbent assay.

As used herein, the term “PCR” means polymerase chain reaction.

As used herein, the term “dI” means deionized.

As used herein, the term “SDS” means sodium dodecyl sulfate.

As used herein, the term “SDS-PAGE” means sodium dodecyl sulfate-polyacrylimide gel electrophoresis.

As used herein, the term “GUS” means β-glucouronidase.

As used herein, the term “PGI” means plant growth inhibition.

As used herein, the term “Ni” refers to nickel.

As used herein, the term “Ni-NTA” refers to nickel sepharose.

As used herein, the term “LB” means Luria-Bertani media.

As used herein, the term “TATA box” refers to a sequence of nucleotides that serves as the main recognition site for the attachment of RNA polymerase in the promoter region of eukaryotic genes. Located at around 25 nucleotides before the start of transcription, it consists of the seven-base consensus sequence TATAAAA, and is analogous to the Pribnow box in prokaryotic promoters.

The term “herbicide,” as used herein, refers to a compound that may be used to kill or suppress the growth of at least one plant, plant cell, plant tissue or seed.

The term “inhibitor,” as used herein, refers to a chemical substance that inactivates the enzymatic activity of DET2. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

The “percent (%) sequence identity” between two polynucleotide or two polypeptide sequences is determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GENEMATCHER PLUS (Paracel, Inc.) (using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “polypeptide” is meant a chain of at least four amino acids joined by peptide bonds. The chain may be linear, branched, circular or combinations thereof.

The polypeptides may contain amino acid analogs and other modifications, including, but not limited to glycosylated or phosphorylated residues.

The term “specific binding” refers to an interaction between DET2 and a molecule or compound, wherein the interaction is dependent upon the primary amino acid sequence or the conformation of DET2.

### Embodiments of the Invention

The present inventors have discovered that inhibition of DET2 gene expression strongly inhibits the growth and development of plant seedlings. Thus, the inventors are the first to demonstrate that DET2 is a target for herbicides.

Accordingly, the invention provides methods for identifying compounds that inhibit DET2 gene expression or activity. Such methods include ligand binding assays, assays for enzyme activity and assays for DET2 gene expression. Any compound that is a ligand for DET2, other than its substrate, (24R)-24-methylcholest-4-en-3-one, may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide, comprising:

a) contacting a DET2 with said compound; and

b) detecting the presence and/or absence of binding between said compound and said DET2, wherein binding indicates that said compound is a candidate for a herbicide.

By “DET2” is meant any enzyme that catalyzes the interconversion of (24R)-24-methylcholest-4-en-3-one with (24R)-24-methyl-5alpha-cholestan-3-one. The DET2 may have the amino acid sequence of a naturally occurring DET2 found in a plant, animal or microorganism, or may have an amino acid sequence derived from a naturally occurring sequence. Preferably the DET2 is a plant DET2. The cDNA (SEQ ID NO:1) encoding the DET2 protein or polypeptide (SEQ ID NO:2) can be found herein as well as in the TIGR database at locus T8P21.4.

By “plant DET2” is meant an enzyme that can be found in at least one plant, and which catalyzes the interconversion of (24R)-24-methylcholest-4-en-3-one with (24R)-24-methyl-5alpha-cholestan-3-one. The DET2 may be from any plant, including both monocots and dicots.

In one embodiment, the DET2 is an Arabidopsis DET2. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis DET2 is from Arabidopsis thaliana.

In various embodiments, the DET2 can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of a DET2 polypeptide may be used in the methods of the invention. The fragments comprise at least 10 consecutive amino acids of a DET2. Preferably, the fragment comprises at least 15, 20, 25, 30, 35, 40, 50, 60, 70, 80, 90 or at least 100 consecutive amino acids residues of a DET2. In one embodiment, the fragment is from an Arabidopsis DET2. Preferably, the fragment contains an amino acid sequence conserved among plant 3-oxo-5-alpha-steroid 4-dehydrogenases. Such conserved fragments are identified in Grima-Pettenuti et al. (1993) Plant Mol Biol 21:1085-1095 and Taveres et al. (2000), supra. Those skilled in the art could identify additional conserved fragments using sequence comparison software.

Polypeptides having at least 80% sequence identity with a plant DET2 are also useful in the methods of the invention. Preferably, the sequence identity is at least 85%, more preferably the identity is at least 90%, most preferably the sequence identity is at least 95% or 99%.

In addition, it is preferred that the polypeptide has at least 50% of the activity of a plant DET2. More preferably, the polypeptide has at least 60%, at least 70%, at least 80% or at least 90% of the activity of a plant DET2. Most preferably, the polypeptide has at least 50%, at least 60%, at least 70%, at least 80% or at least 90% of the activity of the A. thaliana DET2 protein.

Thus, in another embodiment, the invention provides a method for identifying a compound as a candidate for a herbicide, comprising:

a) contacting said compound with at least one polypeptide selected from the group consisting of: a plant DET2, a polypeptide comprising at least ten consecutive amino acids of a plant DET2, a polypeptide having at least 85% sequence identity with a plant DET2, and a polypeptide having at least 80% sequence identity with a plant DET2 and at least 50% of the activity thereof; and

b) detecting the presence and/or absence of binding between said compound and said polypeptide, wherein binding indicates that said compound is a candidate for a herbicide.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a DET2 protein or a fragment or variant thereof, the unbound protein is removed and the bound DET2 is detected. In a preferred embodiment, bound DET2 is detected using a labeled binding partner, such as a labeled antibody. In a variation of this assay, DET2 is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for a herbicide, it can be tested for the ability to inhibit DET2 enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting the presence or absence of a decrease in the growth or viability of said plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be at decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

The ability of a compound to inhibit DET2 activity can be detected using in vitro enzymatic assays in which the disappearance of a substrate or the appearance of a product is directly or indirectly detected. DET2 catalyzes the irreversible reaction of (24R)-24-methylcholest-4-en-3-one to (24R)-24-methyl-5alpha-cholestan-3-one. Methods for detection of (24R)-24-methylcholest-4-en-3-one, and/or (24R)-24-methyl-5alpha-cholestan-3-one include spectrophotometry, mass spectroscopy, thin layer chromatography (TLC) and reverse phase HPLC.

Thus, the invention provides a method for identifying a compound as a candidate for a herbicide, comprising:

a) contacting a (24R)-24-methylcholest-4-en-3-one with DET2;

b) contacting said (24R)-24-methylcholest-4-en-3-one with DET2 and said candidate compound; and

c) determining the concentration of (24R)-24-methyl-5alpha-cholestan-3-one after the contacting of steps (a) and (b).

If a candidate compound inhibits DET2 activity, a higher concentration of the substrate ((24R)-24-methylcholest-4-en-3-one) and a lower level of the product ((24R)-24-methyl-5alpha-cholestan-3-one) will be detected in the presence of the candidate compound (step b) than in the absence of the compound (step a).

Preferably the DET2 is a plant DET2. Enzymatically active fragments of a plant DET2 are also useful in the methods of the invention. For example, a polypeptide comprising at least 100 consecutive amino acid residues of a plant DET 2 may be used in the methods of the invention. In addition, a polypeptide having at least 80%, 85%, 90%, 95%, 98% or at least 99% sequence identity with a plant DET2 may be used in the methods of the invention. Preferably, the polypeptide has at least 80% sequence identity with a plant DET2 and at least 50%, 75%, 90% or at least 95% of the activity thereof.

Thus, the invention provides a method for identifying a compound as a candidate for a herbicide, comprising:

a) contacting (24R)-24-methylcholest-4-en-3-one with a polypeptide selected from the group consisting of: a polypeptide having at least 85% sequence identity with a plant DET2, a polypeptide having at least 80% sequence identity with a plant DET2 and at least 50% of the activity thereof, and a polypeptide comprising at least 100 consecutive amino acids of a plant DET2;

b) contacting said (24R)-24-methylcholest-4-en-3-one with said polypeptide and said compound; and

c) determining the concentration of (24R)-24-methyl-5alpha-cholestan-3-one after the contacting of steps (a) and (b).

Again, if a candidate compound inhibits DET2 activity, a higher concentration of the substrate ((24R)-24-methylcholest-4-en-3-one) and a lower level of the product ((24R)-24-methyl-5alpha-cholestan-3-one) will be detected in the presence of the candidate compound (step b) than in the absence of the compound (step a).

For the in vitro enzymatic assays, DET2 protein and derivatives thereof may be purified from a plant or may be recombinantly produced in and purified from a plant, bacteria, or eukaryotic cell culture. Preferably these proteins are produced using a baculovirus or E. coli expression system. Methods for the purification of 3-oxo-5-alpha-steroid 4-dehydrogenase may be described in Ordman et al. (1991) J Steroid Biochem Mol Biol 39:487-92 and in Quemener et al. (1994) Steroids 59:712-18. Other methods for the purification of DET2 proteins and polypeptides are known to those skilled in the art.

As an alternative to in vitro assays, the invention also provides plant and plant cell based assays. In one embodiment, the invention provides a method for identifying a compound as a candidate for a herbicide, comprising:

a) measuring the expression of DET2 in a plant or plant cell in the absence of said compound;

b) contacting a plant or plant cell with said compound and measuring the expression of DET2 in said plant or plant cell;

c) comparing the expression of DET2 in steps (a) and (b).

A reduction in DET2 expression indicates that the compound is a herbicide candidate. In one embodiment, the plant or plant cell is an Arabidopsis thaliana plant or plant cell.

Expression of DET2 can be measured by detecting the DET2 primary transcript or mRNA, DET2 polypeptide or DET2 enzymatic activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting DET2 RNA include, but are not limited to amplification assays such as quantitative PCR, and/or hybridization assays such as Northern analysis, dot blots, slot blots, in-situ hybridization, transcriptional fusions using a DET2 promoter fused to a reporter gene, DNA assays and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect DET2 protein expression. For detection using gene reporter systems, a polynucleotide encoding a reporter protein is fused in frame with DET2, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107-111), β-galactosidase (Nolan et al. (1988) Proc Natl Acad Sci USA 85:2603-2607), alkaline phosphatase (Berger et al. (1988) Gene 66:10), luciferase (De Wet et al. (1987) Mol Cell Biol 7:725-737), β-glucuronidase (GUS), fluorescent proteins, chromogenic proteins and the like. Methods for detecting DET2 activity are described above.

Chemicals, compounds or compositions identified by the above methods as modulators of DET2 expression or activity can then be used to control plant growth. For example, compounds that inhibit plant growth can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants are grown in Scotts Metro-Mix™ soil (the Scotts Company) or a similar soil mixture in an environmental growth room at 22° C., 65% humidity, 65% humidity and a light intensity of ˜100 β-E m⁻² s⁻¹ supplied over 16 hour day period.

Seed Sterilization

All seeds are surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds are sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ in down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 1-137) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of Antisense Expression Cassettes in a Binary Vector

A fragment or variant of an Arabidopsis thaliana cDNA corresponding to SEQ ID NO:1 was ligated into the PacI/AscI sites of an E. coli/Agrobacterium binary vector in the antisense orientation. This placed transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E.coli. Kanamycin resistant clones were selected and purified. DNA was isolated from each clone and characterized by PCR and sequence analysis. The DNA was inserted in a vector that expresses the A. thaliana antisense RNA, which is complementary to a portion of the DNA of SEQ ID NO:1. This antisense RNA is complementary to the cDNA sequence found in the TIGR database at locus T8P21.4. The coding sequence for this locus is shown as SEQ ID NO:1. The protein encoded by these mRNAs is shown as SEQ ID NO:2.

The antisense expression cassette and a constitutive chemical resistance expression cassette are located between right and left T-DNA borders. Thus, the antisense expression cassettes can be transferred into a recipient plant cell by agroinfection.

### EXAMPLE 3

### Transformation of Agrobacterium with the Antisense Expression Cassette

The vector was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of an Arabidopsis Antisense Target Plants

The antisense expression cassette was introduced into Arabidopsis thaliana wild-type plants by the following method. Five days prior to agroinfection, the primary inflorescence of Arabidopsis thaliana plants grown in 2.5 inch pots were clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L SILWET L-77 (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome in order to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial.

One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% SILWET (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represented stably transformed T1 plants.

### EXAMPLE 5

### Effect of Antisense Expression in Arabidopsis Seedlings

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The transgenic plant line containing the antisense construct exhibited significant developmental abnormalities during early development. Four of ten seedlings were smaller than controls and two of those were chlorotic. Thus, DET2 is essential for normal plant growth and development.

### EXAMPLE 6

### Cloning & Expression Strategies, Extraction and Purification of the DET2 Protein

The following protocol may be employed to obtain the purified DET2 protein.

Cloning and Expression Strategies:

DET2 gene can be cloned into E. coli (pET vectors-Novagen), Baculovirus (Pharmingen) and Yeast (Invitrogen) expression vectors containing His/fusion protein tags. Evaluate the expression of recombinant protein by SDS-PAGE and Western blot analysis.

Extraction:

Extract recombinant protein from 250 ml cell pellet in 3 mL of extraction buffer

By sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni-NTA affinity chromatography (Qiagen).

Purification protocol: perform all steps at 4° C:

Use 3 ml Ni-beads (Qiagen)

Equilibrate column with the buffer

Load protein extract

Wash with the equilibration buffer

Elute bound protein with 0.5 M imidazole

### EXAMPLE 7

### Assays for Testing Inhibitors or Candidates for Inhibition of DET2 Activity

The enzymatic activity of DET2 may be determined in the presence and absence of candidate inhibitors in a suitable reaction mixture, such as described by the following fluorescence assay. An assay mixture is prepared containing 5 ug of cell lysate protein, 0.30-1.0 uM (24R)-24-methylcholest-4-en-3-one, 2 mM NADPH, 0.1% bovine serum albumine, 0.1% NOG (n-octyl-beta-D-glucopyranoside) and 0.1 M sodium phosphate buffer pH 6.8. This reaction mixture is incubated at 37 degrees Celsius for thirty minutes, then the optical density is read at a wavelength of 340 nm. Alternatively, the fluorescence of the reaction can be monitored by utilizing wavelengths of 340 nm (for excitation) and 460 nm (for emission). Quemener et al. (1994) Steroids 59:712-18.

While the foregoing describes certain embodiments of the invention, it will be understood by those skilled in the art that variations and modifications may be made and still fall within the scope of the invention.

What is claimed is:

1. A method for identifying a compound as a candidate for a herbicide, comprising:
a) contacting a DET2 with a compound; and
b) detecting the presence and/or absence of binding between said compound and said DET2, wherein binding indicates that said compound is a candidate for a herbicide.

2. The method of claim 1, wherein said DET2 is a plant DET2.

3. The method of claim 2, wherein said DET2 is an Arabidopsis DET2.

4. The method of claim 3, wherein said DET2 is SEQ ID NO:2.

5. A method for determining whether a compound identified as a herbicide candidate by the method of claim 1 has herbicidal activity, comprising: contacting a plant or plant cells with said herbicide candidate and detecting the presence or absence of a decrease in growth or viability of said plant or plant cells.

6. A method for identifying a compound as a candidate for a herbicide, comprising:
a) contacting a compound with at least one polypeptide selected from the group consisting of: an amino acid sequence comprising at least ten consecutive amino acids of a plant DET2, an amino acid sequence having at least 85% sequence identity with a plant DET2, and an amino acid sequence having at least 80% sequence identity with a plant DET2 and at least 50% of the activity thereof; and
b) detecting the presence and/or absence of binding between said compound and said polypeptide, wherein binding indicates that said compound is a candidate for a herbicide.

7. A method for determining whether a compound identified as a herbicide candidate by the method of claim 6 has herbicidal activity, comprising: contacting a plant or plant cells with said herbicide candidate and detecting the presence or absence of a decrease in growth or viability of said plant or plant cells.

8. A method for identifying a compound as a candidate for a herbicide, comprising:
a) contacting a (24R)-24-methylcholest-4-en-3-one with DET2;
b) contacting said (24R)-24-methylcholest-4-en-3-one with DET2 and a candidate compound; and
c) determining the concentration of at least one of (24R)-24-methylcholest-4-en-3-one, and/or (24R)-24-methyl-5alpha-cholestan-3-one after the contacting of steps (a) and (b).

9. The method of claim 8, wherein said DET2 is a plant DET2.

10. The method of claim 9, wherein said DET2 is an Arabidopsis DET2.

11. The method of claim 10, wherein said DET2 is SEQ ID NO:2.

12. A method for identifying a compound as a candidate for a herbicide, comprising:
a) contacting (24R)-24-methylcholest-4-en-3-one with a polypeptide selected from the group consisting of: a polypeptide having at least 85% sequence identity with a plant DET2, a polypeptide having at least 80% sequence identity with a plant DET2 and at least 50% of the activity thereof, and a polypeptide comprising at least 100 consecutive amino acids of a plant DET2;
b) contacting said (24R)-24-methylcholest-4-en-3-one with said polypeptide and said compound; and
c) determining the concentration of at least one of (24R)-24-methylcholest-4-en-3-one, and/or (24R)-24-methyl-5alpha-cholestan-3-one after the contacting of steps (a) and (b).

13. A method for identifying a compound as a candidate for a herbicide, comprising:
a) measuring the expression of a DET2 in a plant or plant cell in the absence of a compound;
b) contacting a plant or plant cell with said compound and measuring the expression of said DET2 in said plant or plant cell;
c) comparing the expression of DET2 in steps (a) and (b).

14. The method of claim 13 wherein said plant or plant cell is an Arabidopsis plant or plant cell.

15. The method of claim 14, wherein said DET2 is SEQ ID NO:2.

16. The method of claim 13, wherein the expression of DET2 is measured by detecting DET2 mRNA.

17. The method of claim 13, wherein the expression of DET2 is measured by detecting DET2 polypeptide.


SEQUENCE LISTING (2 sequences)

SEQ ID NO: 1 - DNA, 789 bp, Arabidopsis thaliana
  misc_feature: TIGR T8P21.4
atggaagaaatcgccgataaaaccttcttccgatactgtctcctcactcttattttcgccggcccaccaaccgccgtccttctgaaattcctccaagctccttacggtaaacacaaccgtaccggatggggtcccaccgtatctccaccgattgcttggttcgtcatggagagcccaaccttgtggctcactctcctcctcttcccctttggtcgtcacgctctcaaccctaaatctctacttctattctctccttatctcattcattacttccaccgcaccatcatttaccctcttcgcctcttccgcagctccttccccgccggtaaaaacggatttccgatcaccatcgccgccttggctttcacctttaatctcctcaatggttatatccaggcgaggtgggtttcgcattacaaggatgactacgaagacggaaactggttctggtggcggtttgttatcggtatggtggttttcataaccggcatgtatataaatatcacgtcggaccggactttggtacgattgaagaaagagaaccggggaggttatgtgataccgagaggaggctggttcgagttggtaagctgtccgaattattttggagaggcgattgagtggttgggctgggctgttatgacttggtcttgggccggtattggattttttctgtacacgtgttccaatttgtttccgcgtgcacgtgcgagtcacaagtggtacattgccaagttcaaggaagagtatcccaagactcgtaaagctgttattccttttgtgtactga

SEQ ID NO: 2 - protein, 262 aa, Arabidopsis thaliana
MEEIADKTFFRYCLLTLIFAGPPTAVLLKFLQAPYGKHNRTGWGPTVSPPIAWFVMESPTLWLTLLLFPFGRHALNPKSLLLFSPYLIHYFHRTIIYPLRLFRSSFPAGKNGFPITIAALAFTFNLLNGYIQARWVSHYKDDYEDGNWFWWRFVIGMVVFITGMYINITSDRTLVRLKKENRGGYVIPRGGWFELVSCPNYFGEAIEWLGWAVMTWSWAGIGFFLYTCSNLFPRARASHKWYIAKFKEEYPKTRKAVIPFVY